(19) United States
(12) Patent Application Publication
(10) Pub. No.: US 2020/0323660 A1
(43) Pub. Date: Oct. 15, 2020

(54) SYSTEMS AND METHODS FOR QUANTITATIVELY OPTIMIZING THE DESIGN OF PROSTHETIC SOCKETS

(71) Applicant: The Florida State University Research Foundation, Inc., Tallahassee, FL (US)

(72) Inventors: Hiu Wang, Tallahassee, FL (US); Jie Ren, Tallahassee, FL (US)

(73) Assignee: The Florida State University Research Foundation, Inc., Tallahassee, FL (US)

(21) Appl. No.: 16/846,153

(22) Filed: Apr. 10, 2020

Related U.S. Application Data
(60) Provisional application No. 62/832,449, filed on Apr. 11, 2019.

(51) Int. Cl. A61F 2/76; A61F 2/80; A61F 2/50; A61F 2/60; G16H 10/60; G16H 50/70; G16H 50/20; G06F 30/27

(52) U.S. Cl. CPC A61F 2/76; A61F 2/80; A61F 2/5046; A61F 2/60; G06Q 50/04; G16H 50/70; G16H 50/20; G06F 30/27; G16H 10/60

(57) ABSTRACT

Systems and methods for quantitatively optimizing the design of prosthetic sockets are disclosed herein. An example method may involve receiving data indicating a first prosthetic socket design for a first patient and data associated with the first patient. The example method may also involve receiving data indicating a structure of an second prosthetic socket design for a second patient and data associated with the second patient. The example method may also involve determining, based on the data associated with the first patient, the data indicating the structure of the optimized prosthetic socket for the second patient, and the data associated with the second patient, an third prosthetic socket design for the first patient. The example method may also involve providing, based on the third prosthetic socket design for the first patient, an output indicating an area of the first prosthetic socket design to add or remove material.

### FIELD OF THE DISCLOSURE

The disclosure generally relates to prosthetic limb fitment, and more particularly to systems and methods for optimizing the design of prosthetic limb sockets.

### BACKGROUND

In conventional practice, prosthetics may be initially provided for fitment on a patient without performing any pre-fitment data processing to optimize the socket design for the patient's residual limb. This results in a fitment process involving multiple iterations of trial-and-error based testing to determine the optimal prosthetic socket design. Additionally, the final fitted prosthetic socket may not be optimized for the particular patient, but may rather be the best available option. This current method may be both inefficient and costly, as well as unideal for the patient, because of the amount of time the prescribing physician needs to allocate to properly fit the particular patient. Thus, a need exists to optimize this fitment process.

### BRIEF DESCRIPTION OF THE DRAWINGS

The detailed description is set forth with reference to the accompanying drawings. The use of the same reference numerals may indicate similar or identical items. Various embodiments may utilize elements and/or components other than those illustrated in the drawings, and some elements and/or components may not be present in various embodiments. Elements and/or components in the figures are not necessarily drawn to scale. Throughout this disclosure, depending on the context, singular and plural terminology may be used interchangeably.

FIGS. 1A and 1B depict example uses cases of prosthetic socket optimization, in accordance with one or more embodiments of the disclosure.

FIG. 2 depicts a prosthetic socket design optimization system, in accordance with one or more embodiments of the disclosure.

FIG. 3 depicts a prosthetic socket design optimization method, in accordance with one or more embodiments of the disclosure.

FIG. 4 depicts an exemplary block diagram of a dummy residual limb and socket testbed, in accordance with one or more embodiments of the disclosure.

### DETAILED DESCRIPTION

The disclosure is directed to, among other things, systems and methods for optimizing the design of prosthetic sockets. In some embodiments, the systems and methods disclosed herein may more particularly relate to systems and methods for reducing an initial fitment time of a new prosthetic socket for a patient, and may also relate to methods for determining the most optimal individualized socket fit for the patient given the unique physical attributes of the patient, such as their age, height, weight, gait, etc. Although the disclosure herein may primary describe prosthetic sockets, similar systems and methods may apply to orthotic insoles, or any other types of prosthetics as well.

In some embodiments, a conventional initial prosthetic socket fitment process for a patient may involve iterations of trial-and-error fitments of various existing prosthetic sockets of various sizes and shapes to a patient until a prosthetic socket that fits the patient is identified. This may be a time consuming process and the final prosthetic socket that is identified may still not be the optimal prosthetic socket for the patient. That is, the identified prosthetic socket may not be individually tailored for the patient, but may rather be the best available option in terms of fitment. This may result in a prosthetic socket that is functional for the patient, but the suboptimal fit may result in discomfort and/or difficulties in walking. The systems and methods described herein may improve upon this convention process by reducing or eliminating this trial-and-error process and by providing a prosthetic socket that is more individually tailored to the particular patient.

In some embodiments, the systems and methods described herein may accomplish the above by using an artificial intelligence system, such as a machine learning system that may employ transfer learning. Transfer learning may be an approach that leverages previously captured data and learned models of an artificial intelligence system as a starting point for training a new model. That is, the artificial intelligence system may use transfer learning to build upon previous training in order to achieve a better output than if the system were to start fresh with new data. In some embodiments, this transfer learning may be implemented in the systems and methods described herein by taking data captured with respect previous patients that have used the system to obtain an optimized prosthetic socket, and using this data to assist the artificial intelligence system in identifying the optimal prosthetic socket for the current patient. For example, the data may include inputs received for any of the previous patients, such as the height, weight, and/or gait of the previous patients, as well as any other pertinent input data that may have been used to identify the optimal prosthetic sockets for those previous patients. The data may also include the particular prosthetic sockets that were chosen for the previous patients given the input data received for each of the previous patients. That is, data pertaining to the current patient may be received as an input to the artificial intelligence system, and such data may be compared to data of previous patients using transfer learning. For example, as with the previous patients, the data received for the current patient may include, for example, height, weight, limb size and/or shape, gait, etc. In some embodiments, some trial-and-error may still be involved, as some of the data input into the artificial intelligence system for the current patient may involve fitting the current patient with an initial prosthetic socket as additional input data for the artificial intelligence system. For example, fitting the current patient with the initial prosthetic socket may allow for gait information of the current patient to be discerned. In some instances, however, the gait information may be manually input, or may be determined based on any of the other data inputs received with regards to the current patient.

In some embodiments, the artificial intelligence system may receive all of this data as an input and may output an optimal prosthetic socket design for the current patient. The optimal prosthetic socket design may be based on optimality of the socket-limb fit in the patient. This optimality of the prosthetic socket design may further be determined by predicted pressure distribution between the prosthetic socket and the patient's limb. to minimize the pressure peaks that may cause local skin breakdown, blistering, or discomfort, as well as improve any potential gait correction performance as suggested by a prosthetist. Given this, the output of the artificial intelligence system may take one or more of several different forms. For example, one output may include data pertaining to the optimal prosthetic socket design that may be used by a 3D printing system to print the optimal prosthetic socket for the current patient. That is the output of the artificial intelligence system may be provided to the 3D printer, and the 3D printer may use this output to print the optimized prosthetic socket. The data may also be provided to any other type of device that is capable of creating the prosthetic socket. The patient may then be fitted with the prosthetic socket, which may be the optimal prosthetic design for the patient. A second example output may pertain to filling a pre-existing prosthetic socket instead of creating an optimal prosthetic socket (e.g., through 3D printing as described above). In this second example, the output may involve indications of where filler material should be applied to a given pre-existing prosthetic socket (for example, the initial prosthetic socket that is tested on the patient as described above). That is, the pre-existing prosthetic socket may be taken as an input by the artificial intelligence system, and the output may be an indication of the portions of the pre-existing prosthetic socket that may be filled to create the optimal prosthetic socket fit for the current patient. The material used to fill the pre-existing prosthetic socket to create the optimal prosthetic socket for the current patient may include a foam material. In some instances, the filler material may be a flexible and stretchable auxetic foam treated by carbon nanotube solutions or traditional polyester foams. Filling the pre-existing prosthetic socket may effectively convert the pre-existing prosthetic socket into the optimized prosthetic socket without having to create an entirely new prosthetic socket for the patient.

In some embodiments, the optimization of the prosthetic socket for the current patient may extend beyond the initial fitment process as described above as well. That is, the artificial intelligence system may continue to monitor data pertaining to the optimal prosthetic socket as being used by the current patient after the initial fitment. This continued monitoring may be performed, for example, through an application on a mobile device of the patient that may receive data from, for example, sensor associated with the prosthetic and communicate this data to the artificial intelligence system. In some instances, the artificial intelligence system may be located remotely to the application, and in some instances the artificial intelligence system may be integrated into the application. For example, the artificial intelligence system may continue to monitor factors such as gait of the current patient, identified comfort levels, etc. Such information may be obtained by the artificial intelligence system in a number of different methods. In one example method, the information may be obtained through sensors embedded in the prosthetic socket. For example, the sensors may be integrated into the foam material that was added to the prosthetic socket, or may be added to the prosthetic socket itself. In a second example, method, the information may be manually provided by the patient. For example, the patient may manually provide information about their gait, level of comfort, etc. In some embodiments, the artificial intelligence system may also continue to receive inputs from other patients being fitted with prosthetic sockets, and may further refine its model through transfer learning of the data obtained from such patients. Based on all of these data inputs, the artificial intelligence system may identify a new optimal prosthetic socket design for the current patient. The optimal prosthetic socket design may be based on optimality of the socket-limb fit in the patient. This optimality of the prosthetic socket design may further be determined by predicted pressure distribution between the prosthetic socket and the patient's limb. to minimize the pressure peaks that may cause local skin breakdown, blistering, or discomfort, as well as improve any potential gait correction performance as suggested by a prosthetist. The current patient may then opt to either have the new optimal prosthetic design created, for example through 3D printing, or may have the filling in the prosthetic socket they are currently using adjusted and/or may have more filling added to result in the new optimal prosthetic socket determined by the artificial intelligence system. This may serve to ensure real-time optimization of the patient's prosthetic socket as the patient's needs may change (for example if their physical attributes are to change) and because the artificial intelligence system may develop better models as more information from more patients is gathered.

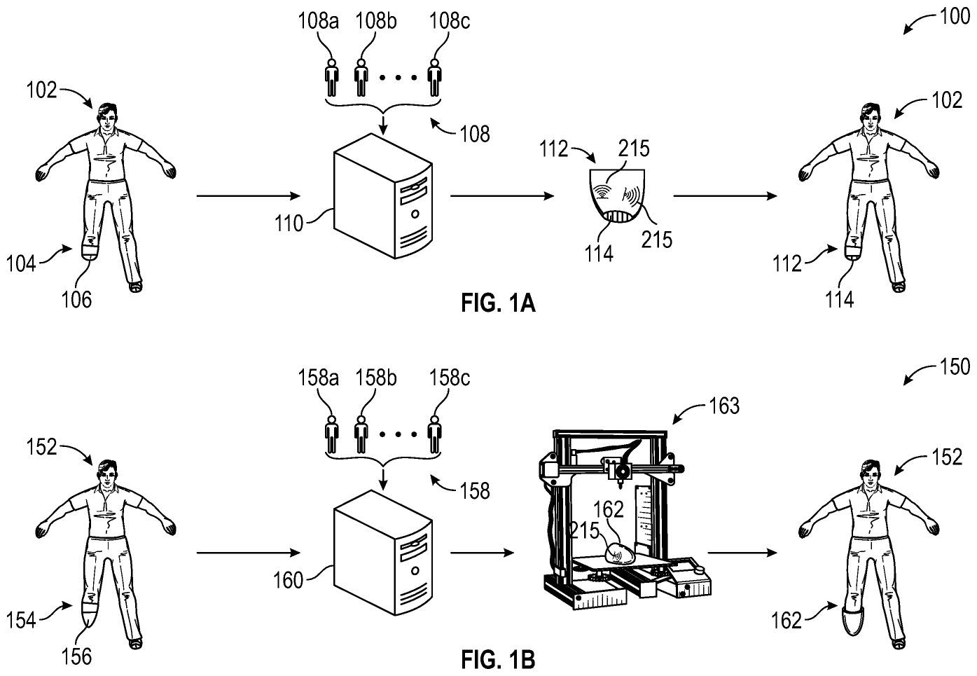

Turning to the figures, FIGS. 1A and 1B depict example use cases of the systems and methods described herein. In some embodiments, FIG. 1A may depict a use case 100 for creating an optimal prosthetic socket for a patient by filling an initial prosthetic socket fitted to the patient with filler material, such as foam. The use case 100 may begin with a current patient 102 being fitted with an initial prosthetic socket 104. The current patient 102 may be the patient seeking to have an optimized prosthetic socket created for them. The initial prosthetic socket 104 may not be optimized for fitment to the current patient 102, but may instead be a pre-existing prosthetic socket that is determined to be a closest fit for the current patient 102 out a set of pre-existing prosthetic sockets. As such, the initial prosthetic socket 104 may include one or more non-conforming regions 106. A non-conforming region 106 may refer to a portion of the initial prosthetic socket 104 that does not optimally conform to the residual limb of the current patient 102. The initial prosthetic socket 104 including such non-conforming regions 106 may cause discomfort for the current patient 102 when walking using the initial prosthetic socket 104, and may also hinder the gait of the current patient 102 (e.g., the ability of the current patient 102 to properly walk with the initial prosthetic socket 104).

In some embodiments, in order to remedy the aforementioned suboptimal fitment between the initial prosthetic socket 104 and the current patient 102, the use case 100 may then involve providing inputs to a prosthetic socket design optimization system 110, which may be an artificial intelligence system that uses transfer learning as described above (the terms “artificial intelligence system” and “prosthetic design optimization system” may be used interchangeably herein). In some embodiments, the inputs may include data about a structure of the initial prosthetic socket 104, including for example, the size, shape, material, weight, and any other physical properties of the initial prosthetic socket 104. The inputs may also include information about the current patient 102, such as the height, weight, and/or any other physical properties of the current patient 102, including the physical shape of a residual limb of the current patient to which the initial prosthetic socket 104 is fitted. The inputs may also include information about the gait of the current patient 102 with the initial prosthetic socket 104 fitted. The gait of the current patient 102, for example, may refer to the manner in which the current patient 102 walks with the initial prosthetic socket 104 fitted. The inputs may also include data regarding the interaction between the current patient 102 and the initial prosthetic socket 104. The interaction, for example, may involve the level of physical conformity of the initial prosthetic socket 104 to the residual limb of the current patient 102. For example, there may exist one or more non-conforming regions 106 between the prosthetic socket 104 and the current patient 102. In some instances, the non-conformity between the socket and limb of the current patient 102 may be estimated by determining the peak pressure at certain areas between the initial prosthetic socket 104 and the current patient 102, as well as overall pressure non-uniformity between the limb-socket interface of the current patient 102 when the current patient's 102 body weight is applied. The non-conformity may also be estimated by determining a deviation of an actual gait performance measure with the current patient 102 fitted with the initial prosthetic socket 104 from the desired pattern of a certain gait performance measure for the current patient 102 (e.g., the curving pattern of a Center of Plantar Pressure line). The interaction may also include an indication of a comfort level of the current patient 120 with the initial prosthetic socket 104 fitted, for example, based on the level of pressure between the initial prosthetic socket 104 and the current patient 102 at particular areas of the initial prosthetic socket 104. In some instances, this interaction information may be determined based on sensors fitted to the initial prosthetic socket 104. In some instances, the interaction information may be manually provided by the current patient 102. The inputs may also include any other information about the current patient 102, the initial prosthetic socket 104, or the interaction between the two. Additionally, in some instances a dummy residual limb that is a replica model of the current patient's residual limb may be created for fitment to the initial prosthetic socket 104 to eliminate the need for the current patient 102 to undergo the initial fitment testing. The dummy residual limb may be created by 3D scanning the residual limb of the current patient 102, for example.

In some embodiments, in addition to receiving inputs regarding the initial prosthetic socket 104 and the current patient 102, prosthetic socket design optimization system 110 may also receive inputs associated with one or more previous patients 108 (e.g., previous patient 108a, previous patient 108b, previous patient 108c, or any other number of previous patients 108). Previous patients 108 may refer to patients that have already undergone the systems and methods described herein and have already received optimized prosthetic sockets. The inputs from the previous patients 108 may include any of the data described above as being received with respect to the current patient 102. The inputs from the previous patients 108 may also include the final design of the optimized prosthetic socket that was selected for each of the previous patients 108 based on their own individual undergone prosthetic socket optimizes processes. In some embodiments, receiving the inputs from the one or more previous patients 108 may refer to the prosthetic socket design optimization system 110 using transfer learning to incorporate the knowledge obtained from performing the processes with the previous patients 108 in determining the optimized prosthetic socket design for the current patient 102. The transfer learning may be implemented by identifying a common data structure among the data from previous patients 108 and/or the current patient 102 and leveraging such data commonality to provide supplemental information to the prosthetic socket design optimization system 110. Specifically, this learning strategy may introduce to the objective function in learning the gait progression model a function (e.g., a norm) that may force the outcome of the learning to have a certain level of closeness to a pre-determined common structure in the pressure/gait data from previous patients 108. The prosthetic socket design optimization system 110 may compare a number of candidate options for the common data structure and the norm function based on the current patient's 102 data to make a final selection.

In some embodiments, the prosthetic socket design optimization system 110 may take some or all of the inputs described above and may output an optimized prosthetic socket design 112 for the current patient 102. In the use case 100 described with respect to FIG. 1A, the optimized prosthetic socket design 112 may be the initial prosthetic socket 104 with filler material included to mitigate or eliminate the nonconforming regions 106 and also to help increase the comfort of the current patient 102 in using the prosthetic socket. As such, the output may include indications of one or more filler areas 114, in which a filler materials should be added to the initial prosthetic socket 104 to result in the optimized prosthetic socket design 112. The output may also similarly include one or more areas where material may be removed. The filler material may be, for example, flexible and stretchable auxetic foam treated by carbon nanotube solutions or traditional polyester foams. The foam material may also include sensing capabilities, such that the foam material may provide sensory feedback as to interactions between the residual limb of the current patient 102 and the optimized prosthetic socket. For example, the foam material may include pressure sensors, such as electrical resistance sensor arrays embedded within an auxetic foam, where the auxetic foam can expand its volume under load and be compliant to the limb shape, capable of sensing the pressure at various points of contact between the residual limb and the prosthetic socket, as well as gait information regarding the current patient 102. Finally, in some embodiments, the use case 100 may involve fitting the optimized prosthetic socket design 112 to the current patient 102. It should be noted that although use case 100 (as well as the remainder of the disclosure provided herein) may refer to adding filler material to an initial prosthetic socket, material may also be removed in creating the optimized prosthetic socket as well.

FIG. 1B illustrates a use case 150 for providing an optimized prosthetic socket to a patient. The elements depicted in FIG. 1B may be similar to elements depicted in FIG. 1A. For example, current patient 152 may correspond to current patient 102, initial prosthetic socket 154 may correspond to initial prosthetic socket 104, nonconforming region 106 may correspond to non-conforming region 156, previous patients 158a, 158b, and/or 158c may correspond to previous patients 108a, 108b, and/or 108c, and so on. However, the use case 150 may differ from use case 100 depicted in FIG. 1A in that instead of providing an indication of areas to add filler material to an initial prosthetic socket 154, a new prosthetic socket 162 that is the same structural design as the optimized prosthetic socket determined by the prosthetic socket design optimization system 160 may be created. For example, the prosthetic socket design optimization system 160 may output data regarding an optimized prosthetic socket 162, and such data may be used by a 3D printing device 163 to print a copy of the optimized prosthetic socket 162. The optimized prosthetic socket 162 may then be fitted to the current patient 152 without the need to add any filler material (however, filler material may still be added if necessary).

In some embodiments, the optimization process may extend beyond the use case 100 and use case 150. That is, optimization may continuously be performed after the initial fitment of the optimized prosthetic socket (e.g., optimized prosthetic socket 112 with respect to FIG. 1A and optimized prosthetic socket 162 with respect to FIG. 1B). This may allow the prosthetic socket of the current patient (e.g., current patient 102 with respect to FIG. 1A and current patient 152 with respect to FIG. 1B) to be continuously altered and optimized if it is determined that a more optimal prosthetic socket design exists. Such a determination may be based on additional transfer learning by the prosthetic socket design optimization system (e.g., prosthetic socket design optimization system 110 with respect to FIG. 1A and prosthetic socket design optimization system 160 with respect to FIG. 1B) as the processes are applied to additional patients. Such a determination may also be made on additional data received regarding the interaction between the optimized prosthetic socket and the current patient (for example, through data captured by the foam material as described above). This may be accomplished using an application on a mobile device of the current patient 102 and/or current patient 104 that may receive the sensory data and provide the data to the prosthetic socket design optimization system (e.g., prosthetic socket design optimization system 110 with respect to FIG. 1A and prosthetic socket design optimization system 160 with respect to FIG. 1B).

### Prosthetic Socket Design Optimization System

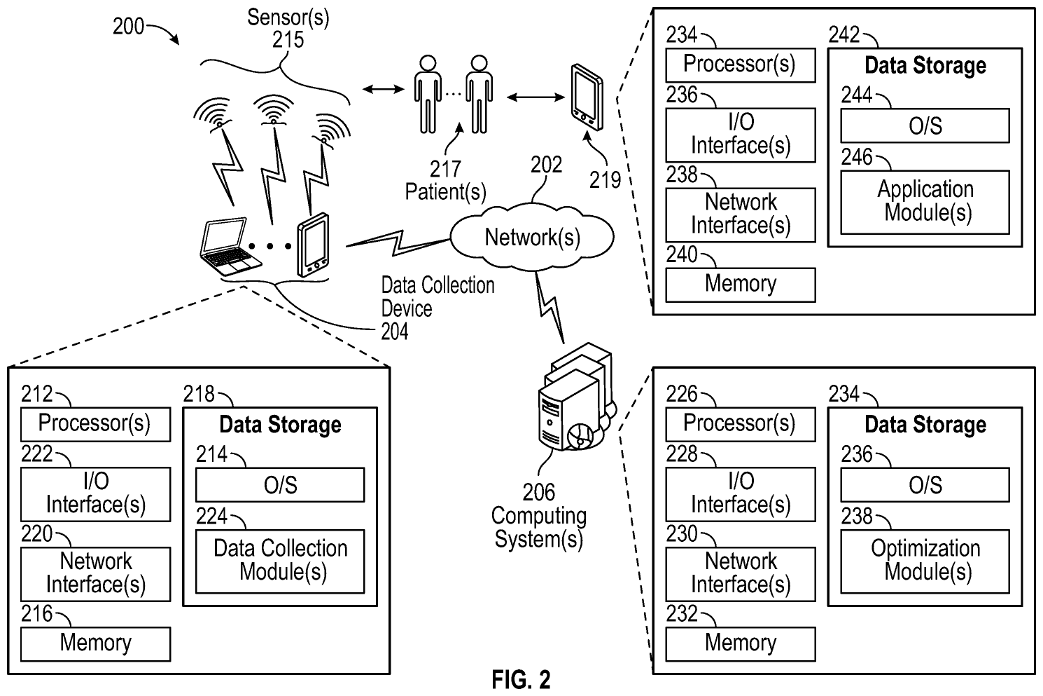

FIG. 2 depicts an illustrative system 200 for a prosthetic socket design optimization system in which techniques and structures for providing the systems and methods disclosed herein may be implemented. The illustrative system 200 may include one or more computing systems 206, one or more networks 202, and one or more data-collection devices 204. The system 100 may interact with one or more patients 217 in communication with the one or more data collection devices 204 and/or one or more sensors 215. The one or more patients 217 may also be associated with one or more mobile devices 219. The one or more data collection devices 204 may also be in communication with the one or more sensors 215. In some instances, one or more of the sensors 215 may be located in the data collection devices 204. In some instances, the sensors 215 may also be located within a prosthetic socket as described above.

Any of the components of the system 200, such as the data collection devices 204, computing systems 206, and/or mobile devices 219 may be configured to communicate with each other and any other component of the system 200 via one or more networks 202. A network 202 may include, but is not limited to, any one or a combination of different types of suitable communications networks such as, for example, cable networks, public networks (e.g., the Internet), private networks, wireless networks, cellular networks, or any other suitable private and/or public networks. Further, the network 202 may have any suitable communication range associated therewith and may include, for example, global networks (e.g., the Internet), metropolitan area networks (MANs), wide area networks (WANs), local area networks (LANs), or personal area networks (PANs). In addition, the network 202 may include any type of medium over which network traffic may be carried including, but not limited to, coaxial cable, twisted-pair wire, optical fiber, a hybrid fiber coaxial (HFC) medium, microwave terrestrial transceivers, radio frequency communication mediums, satellite communication mediums, or any combination thereof.

In some embodiments, a data collection device 204 may comprise one or more processors 212, one or more I/O interfaces 222, one or more network interfaces 220, memory 216, data storage 218, an operating system (O/S) 214, and one or more data collection modules 224.

The data collection device 204 may include one or more processors 212 that may include any suitable processing unit capable of accepting digital data as input, processing the input data based on stored computer-executable instructions, and generating output data. The computer-executable instructions may be stored, for example, in the data storage 218 and may include, among other things, operating system software and application software. The computer-executable instructions may be retrieved from the data storage 218 and loaded into the memory 216 as needed for execution. The processor 212 may be configured to execute the computer-executable instructions to cause various operations to be performed. Each processor 212 may include any type of processing unit including, but not limited to, a central processing unit, a microprocessor, a microcontroller, a Reduced Instruction Set Computer (RISC) microprocessor, a Complex Instruction Set Computer (CISC) microprocessor, an Application Specific Integrated Circuit (ASIC), a System-on-a-Chip (SoC), a field-programmable gate array (FPGA), and so forth.

The data storage 218 may store program instructions that are loadable and executable by the processors 212, as well as data manipulated and generated by one or more of the processors 212 during execution of the program instructions. The program instructions may be loaded into the memory 216 as needed for execution. Depending on the configuration and implementation of each of the data collection devices 204, the memory 216 may be volatile memory (memory that is not configured to retain stored information when not supplied with power) such as random access memory (RAM) and/or non-volatile memory (memory that is configured to retain stored information even when not supplied with power) such as read-only memory (ROM), flash memory, and so forth. In various implementations, the memory 216 may include multiple different types of memory, such as various forms of static random access memory (SRAM), various forms of dynamic random access memory (DRAM), unalterable ROM, and/or writeable variants of ROM such as electrically erasable programmable read-only memory (EEPROM), flash memory, and so forth.

Various program modules, applications, or the like may be stored in data storage 218 that may comprise computer-executable instructions that when executed by one or more of the processors 212 cause various operations to be performed. The memory 216 may have loaded from the data storage 218 one or more operating systems (O/S) 214 that may provide an interface between other application software (e.g., dedicated applications, a browser application, a web-based application, a distributed client-server application, etc.) executing on the data collection device 204 and the hardware resources of the data collection device 204. More specifically, the O/S 214 may include a set of computer-executable instructions for managing the hardware resources of the data collection devices 104 and for providing common services to other application programs (e.g., managing memory allocation among various application programs). The O/S 214 may include any operating system now known or which may be developed in the future including, but not limited to, any mobile operating system, desktop or laptop operating system, mainframe operating system, or any other proprietary or open-source operating system.

The data storage 218 may additionally include various other program modules that may include computer-executable instructions for supporting a variety of associated functionality. For example, the data storage 218 may include one or more data collection modules 224. In the embodiment shown, a data collection module 224 can include computer-executable instructions that in response to execution by one or more processors 212 cause operations to be performed including capturing data relating to an interaction between the patient 217 and the initial prosthetic socket (for example, as described above with respect to FIGS. 1A and 1B). For example, capturing data may include identifying non-conforming areas of the prosthetic socket with respect to the patient's residual limb. The non-conforming areas may be identified by checking a peak pressure and a non-uniformity of pressure distribution between the initial prosthetic socket and the patient 217 as follows. The pressure readings from various sensors may be collected at different times to obtain a pressure distribution color map. The variation of the pressure distribution and peak values at different times may be used to determine if the socket provides sufficient cushion to the limb of the patient 217. A large pressure peak or variation in a region may indicate that a non-conforming area may exist surrounding the region. Capturing data may also include determining a gait of the patient 217 while fitted with the initial prosthetic socket. The operations may also include capturing data beyond the interaction between the initial prosthetic socket and the patient 217. For example, data may be captured regarding the patient 217 themselves, such as their height, weight, physical features, etc., as well as the size, shape, and other features of the patient's 217 residual limb itself. Additionally, any other type of data may be captured, such as the data described below with respect to the sensors 215. The data described as being captured may be captured by the one or more sensors 215 as described below.

The data collection device 204 may further include one or more network interfaces 220 that facilitate communication between the computing system 206 and other devices of the illustrative system 200 or application software via the network 202. The data collection device 204 may additionally include one or more respective input/output (I/O) interfaces 222 (and optionally associated software components such as device drivers) that may support interaction between a user and a variety of I/O devices, such as a keyboard, a mouse, a pen, a pointing device, a voice input device, a touch input device, gesture detection or input device, a display, speakers, a camera, a microphone, a printer, and so forth.

The data collection module 224 may receive data from the one or more sensors 215. The one or more sensors 215 may comprise accelerometers, gyroscopes, pressure sensors, temperature sensors, and/or any other type of sensor, . These sensors 215 may collect data from a patient 217 with an initial prosthetic socket, where the data may include movement speed, orientation of the prosthetic socket and limb, and pressure at various points throughout the socket that are in contact with the patient's 217 residual limb, or any other data relevant to fitment of a prosthetic socket or gait determinations. In some embodiments, the data collected may also relate to the patient's 217 residual limb itself, including size, shape, or any other relevant data points. In some embodiments, data may be collected for one or two iterations of prosthetic socket designs being fitted to the patient's residual limb. In some embodiments, the data may be collected from a dummy residual limb and socket that is personalized for the patient, or any other dummy residual limb and socket. The data collection module 224 may then transmit the collected data to the computing system 206. In some embodiments, the transmission may include any of the data collected from any of the aforementioned sensors, and/or may also include the most recently-tested prosthetic socket design tested on the patient 217 and/or the most recently created and tested dummy residual limb and socket for the patient 217. The collected data from the patient's 217 residual limb may be converted by the data collection module 224 to a computer model with meshes (*.stl file) after data cleaning. The meshed model may be the primary format of data storage containing the information of spatial data for the analysis on the computing system and/or local 3D printing of test prosthetic socket.

In some embodiments, the computing system 206 may comprise one or more processors 226, one or more prosthetic socket optimization modules 238, an operating system (O/S) 236, one or more network interfaces 230, one or more I/O interfaces 228, memory 232, and data storage 234. The one or more processors 226, operating systems 236, one or more network interfaces 230, one or more I/O interfaces 228, and memory 232 may be similar to those found in the data-collection devices 204.

The computing system 206 may include one or more processors 226 that may include any suitable processing unit capable of accepting digital data as input, processing the input data based on stored computer-executable instructions, and generating output data. The computer-executable instructions may be stored, for example, in the data storage 234 and may include, among other things, operating system software and application software. The computer-executable instructions may be retrieved from the data storage 234 and loaded into the memory 232 as needed for execution. The processor 226 may be configured to execute the computer-executable instructions to cause various operations to be performed. Each processor 226 may include any type of processing unit including, but not limited to, a central processing unit, a microprocessor, a microcontroller, a Reduced Instruction Set Computer (RISC) microprocessor, a Complex Instruction Set Computer (CISC) microprocessor, an Application Specific Integrated Circuit (ASIC), a System-on-a-Chip (SoC), a field-programmable gate array (FPGA), and so forth.

The data storage 234 may store program instructions that are loadable and executable by the processors 226, as well as data manipulated and generated by one or more of the processors 226 during execution of the program instructions. The program instructions may be loaded into the memory 232 as needed for execution. Depending on the configuration and implementation of each of the computing systems 206, the memory 232 may be volatile memory (memory that is not configured to retain stored information when not supplied with power) such as random access memory (RAM) and/or non-volatile memory (memory that is configured to retain stored information even when not supplied with power) such as read-only memory (ROM), flash memory, and so forth. In various implementations, the memory 232 may include multiple different types of memory, such as various forms of static random access memory (SRAM), various forms of dynamic random access memory (DRAM), unalterable ROM, and/or writeable variants of ROM such as electrically erasable programmable read-only memory (EEPROM), flash memory, and so forth.

Various program modules, applications, or the like may be stored in data storage 234 that may comprise computer-executable instructions that when executed by one or more of the processors 226 cause various operations to be performed. The memory 232 may have loaded from the data storage 234 one or more operating systems (O/S) 236 that may provide an interface between other application software (e.g., dedicated applications, a browser application, a web-based application, a distributed client-server application, etc.) executing on the computing system 206 and the hardware resources of the data collection device 204. More specifically, the O/S 236 may include a set of computer-executable instructions for managing the hardware resources of the data collection devices 204 and for providing common services to other application programs (e.g., managing memory allocation among various application programs). The 0/5 236 may include any operating system now known or which may be developed in the future including, but not limited to, any mobile operating system, desktop or laptop operating system, mainframe operating system, or any other proprietary or open-source operating system.

The data storage 234 may additionally include various other program modules that may include computer-executable instructions for supporting a variety of associated functionality. For example, the data storage 234 may include one or more prosthetic socket optimization modules 238. In some embodiments, the prosthetic socket optimization module 238 may include transfer learning artificial intelligence. The prosthetic socket optimization module 238 may perform operations including receiving data indicating a first prosthetic socket design for a first patient and data associated with the first patient. The operations may also include receiving data indicating a structure of an second prosthetic socket design for a second patient and data associated with the second patient. The operations may also include determining, based on the data associated with the first patient, the data indicating the structure of an optimized prosthetic socket for the second patient, and the data associated with the second patient, an third prosthetic socket design for the first patient. The operations may also include providing, based on the third prosthetic socket design for the first patient, an output indicating an area of the first prosthetic socket design to add or remove material. The operations may also include any other operations described herein (for example, any other methods described herein).

The data storage 234 may also store data collected from one or more patients. In some embodiments, the data collected may pertain to the observed gait of a patient testing a prosthetic limb fitment. The collected data may include pressure distribution data from pressure sensor arrays and/or orientation and acceleration data from accelerometer/gyroscope sensors. The data may be saved as cyclic time series corresponding to different locations of the sensors. The data may also comprise additional patient information such as the history and/or physical attributes of a patient (height, weight, body structure, etc.). Any time data is received from a data-collection device 208 regarding a new patient, the data may then be stored in patient data storage 240 with the data for the one or more other patients. This data may then be used to compare against data of subsequent patients in the future.

The computing system 206 may further include one or more network interfaces 230 that facilitate communication between a computing system 206 and other devices of the illustrative system 200 or application software via the network 202. The computing system 206 may additionally include one or more respective input/output (I/O) interfaces 228 (and optionally associated software components such as device drivers) that may support interaction between a user and a variety of I/O devices, such as a keyboard, a mouse, a pen, a pointing device, a voice input device, a touch input device, gesture detection or input device, a display, speakers, a camera, a microphone, a printer, and so forth.

The one or more mobile devices 219 may also include similar elements as the data collection device(s) 204 and/or the computing system(s) 206, such as one or more processor(s) 234, I/O interface(s) 236, network interface(s) 238, memory 240, data storage 242, O/S 244, and/or one or more modules, such as an application module 246. The one or more application modules 246 may be capable of capturing data regarding an optimized prosthetic socket subsequent to a patient being fitted with the optimized prosthetic socket as described herein. The application module 246 may also be capable of performing any of the other operations described herein (for example, operations performed by the data collection device(s) 204 and/or the computing system(s) 206).

It should be noted that while systems and methods disclosed herein may refer to prosthetic limbs, prosthetic sockets, and the like, such systems and methods may be extended to the application of designing orthotic insoles that aim to correct gait problems during walking. Such systems and methods may also be extended to any other application involving corrective mechanisms for various gait problems in patients.

### Prosthetic Socket Design Optimization Method

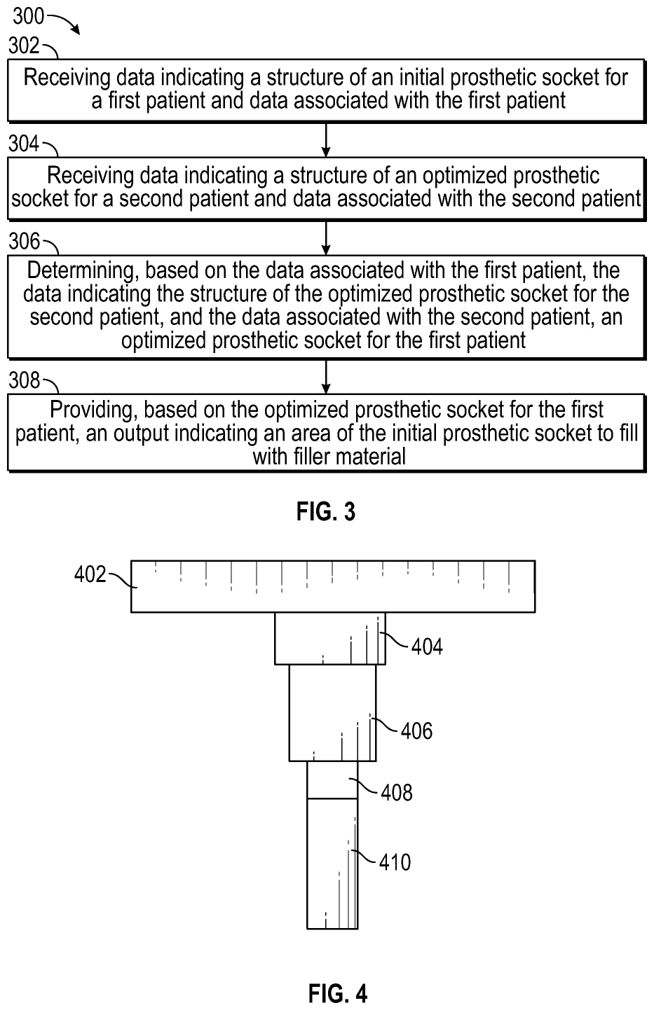

FIG. 3 depicts an example method 300. In some instances, the method 300 may be implemented at the prosthetic socket design optimization system (e.g., the computing system 206 with respect to FIG. 2). In various embodiments, at block 302, the method may involve receiving data indicating a first prosthetic socket design for a first patient and data associated with the first patient. In some embodiments, the first prosthetic socket design may be an initial prosthetic socket for fitment to a current patient. Additionally, the first patient may be the current patient being fitted with the initial prosthetic socket. The initial prosthetic socket, as described above, may be a socket that the first patient is initially fitted with, and may serve as a starting point that may ultimately result in the optimized prosthetic socket for the first patient. That is, the initial prosthetic socket may serve as a baseline prosthetic socket to be modified to create the optimized prosthetic socket. The initial prosthetic socket may also be used for data collection purposes, such as to determine a gait of the first patient and/or interactions between the initial prosthetic socket and the residual limb of the first patient as described below. The data indicating the structure of the initial prosthetic socket may include structural information regarding the initial prosthetic socket, such as, for example, a size and/or shape of the initial prosthetic socket, material, etc. Additionally, the data associated with the first patient may refer to any data described above as being received for a current patient (e.g., the patient being fitted for an optimized prosthetic socket). For example, such data may include physical attributes of the first patient, such as their height, weight, residual limb shape and/or size, etc. The data may also include a medical history of the first patient. The data may also include a gait of the first patient, which may be determined after fitting the first patient with the initial prosthetic socket. Furthermore, data regarding an interaction between the current patient and the initial prosthetic socket may be received. Such an interaction may refer to, for example, information regarding points or contact (or lack thereof) between a residual limb of the current patient and the initial prosthetic socket. Such information may include, for example, information regarding pressure values at various points of contact between the residual limb and the prosthetic socket, as well as any other pertinent information.

In some embodiments, the initial prosthetic socket may be a prosthetic socket selected from a set of pre-existing prosthetic sockets. That is, a trial-and-error process may initially be used to identify the initial prosthetic socket to fit to the first patient. In some embodiments, however, the initial prosthetic socket may instead be created instead of choosing an initial prosthetic socket from a pre-existing set of prosthetic sockets. For example, the data obtained regarding the residual limb may be sent to a device, such as a computer, for modeling of the initial prosthetic socket. The data may then be used to create the initial prosthetic limb. For example, the device may send the data to a 3D printer. The 3D printer may then 3D print the initial prosthetic socket based on the data. In some embodiments, the data may be sent directly to the 3D printer for printing. In some embodiments, any other method of creating the initial prosthetic socket may be utilized, such as using a molding plaster or CAD-CAM system. In some embodiments, the initial prosthetic socket may be created based on data available or obtained from the first patient regarding their medical and/or other history and/or physical attributes such as height, weight, body structure, etc. In some embodiments, the initial prosthetic socket may be created based on a combination of any of the aforementioned data, or any other relevant data.

In some embodiments, as described above, gait data may be captured for the first patient after fitting the first patient with the initial prosthetic socket. Gait data may refer to the manner in which the first patient walks or achieves locomotion through the movement of human limbs. The gait data may be captured using any data capture and/or processing device capable of capturing data. In some instances, the gait data may be captured using an Arduino or similar microcontroller. The data capture and/or processing device may be in communication with one or more sensors, including one or more gyroscopes, one or more accelerometers, and/or one or more pressure sensors. These sensors may be attached to, or in the vicinity of, the initial prosthetic socket fitted to the first patient, and may capture data as the patient performs actions wearing the prosthetic. Specifically, the sensors may capture data regarding the movement speed and orientation of the prosthetic socket and accompanying prosthetic limb, as well as the magnitude of the pressure at certain points of contact between the first patient's residual limb and the prosthetic socket. In some embodiments, a limited number of iterations of initial prosthetic socket design and testing may be performed on the first patient. For example, two iterations of initial prosthetic socket design and testing may be performed on the first patient. In some embodiments, beyond these two iterations, further testing may be performed on a dummy residual limb (e.g., 3D model of the first patient's residual limb that may be created based on the data regarding the first patient's residual limb) and socket created for the first patient, and/or may be performed by the prosthetic socket optimization module 238. This may serve the purpose of eliminating any undue and excessive testing on the first patient.

In some embodiments, any of the above-mentioned data may be send to a prosthetic socket optimization system (e.g., computing system 206 described with reference to FIG. 2). The prosthetic socket optimization system may already have stored gait data and/or optimized prosthetic socket designs, and/or residual limb data, and/or any other relevant data for one or more other patients. For example, the gait data and/or optimized prosthetic socket design of one or more other patients may include data captured from previous patients seeking prosthetic sockets, as well as determined optimized prosthetic socket designs for those other patients based on at least their gait data and/or any other received data associated with those other patients. The prosthetic socket optimization system may use the data to determine an optimized prosthetic socket for the first patient as described in the subsequent blocks of method 300 described below. The prosthetic socket optimization system may also store the data in order to apply the same transfer learning principles used to determine the optimized prosthetic socket for the first patient (e.g., the current patient) to any future patients that may seek to obtain a optimized prosthetic socket using the systems and methods described herein.

In various embodiments, at block 304, the method may involve receiving data indicating a structure of an second prosthetic socket design for a second patient and data associated with the second patient. In some embodiments, the second patient may be a previous patient as described above, and the second prosthetic socket design may be an optimal prosthetic socket that was created for the previous patient. The data indicating the structure of the optimized prosthetic socket for the second patient and data associated with the second patient may include some of the same as the data indication the structure indicating the structure of the optimized prosthetic socket for the first patient and data associated with the first patient. However, the prosthetic socket structural data for the second patient may be data regarding an optimized prosthetic socket instead of an initial prosthetic socket as was described with respect to the first patient. This may be because the first patient may be a current patient seeking an optimized prosthetic socket and the second patient may be a previous patient that has already received an optimized prosthetic socket using the systems and methods described herein.

In various embodiments, at block 306, the method may involve determining, based on the data associated with the first patient, the data indicating the structure of an optimized prosthetic socket for the second patient, and the data associated with the second patient, an third prosthetic socket design for the first patient. In some embodiments, the third prosthetic socket design may be an optimized prosthetic socket design for the current patient. In some instances, determining the optimized prosthetic socket for the first patient may involve providing the above input information to the prosthetic socket optimization system (e.g., the computing system 206 described with respect to FIG. 2). The prosthetic socket optimization system may use the input information to determine similarities between the data for the first patient and the data for the second patient (which, as described above, may be data pertaining to a previous patient). In some embodiments, similarities may also be determined between the data for the first patient and the data for a dummy residual limb and socket created for the first patient. The prosthetic socket optimization system may also compare any other relevant data, such as the first and other patient's medical and/or other history and/or physical attributes such as height, weight, body structure, etc. This, and any other, comparison may be performed using a machine-learning algorithm, and/or transfer-learning algorithm, and/or any other artificial intelligence algorithm or method of comparison. The prosthetic socket optimization system may also store the gait data, and any other relevant data, of the first patient with the gait data, and any other relevant data, of the second patient as to perform similar operations for use with subsequent patients (e.g., a future patient comes in requiring a prosthetic socket and goes through the same operations described herein, but the prosthetic socket optimization system now includes the data received from the first patient as well). In some embodiments, any of the data mentioned herein may be stored locally on a device that is not connected to the cloud, and/or may also be stored on a remote server.

In some embodiments, the prosthetic socket performance model may be in the form of a mathematical equation indicating how the prosthetic socket design would perform if fitted to the first patient. To make this determination, the optimization module 238 of the computing system 206 may iteratively test models of prosthetic socket designs (these prosthetic socket designs may be explored by the optimization module 238 itself, as opposed to being inputs in the form of prosthetic socket designs based on prosthetic sockets physically tested on a patient) and produce outputs at least in the form of prosthetic socket performance models for one or each of the prosthetic socket design (an output may also include the prosthetic design model itself). For example, the output from the prosthetic socket performance model can be the predicted pressure distribution, magnitude/location of the peak pressure, the curving pattern of a center of plantar pressure gait line, and any other metric suggested by a prosthetist. The prosthetic socket designs may be determined by running metaheuristic algorithm, such as an evolutionary algorithm (EA) or simulated annealing, based on the pressure or gait pattern prediction learned by the transfer learning algorithm. Additionally, a transfer learning algorithm may be used to identify a common data structure among all patients' pressure and gait data (e.g., previous patients and/or the current patient). Such a common structure may determine the relatedness or similarity among the patients (e.g., previous patients and/or the current patient), thereby providing a way of supplementing information to the learning in AI for the current patient with limited data. The format of the common data structure and learning algorithm to determine the structure may be selected from a number of candidate options based on comparison of the transfer learning performance for the patients' data.

The above-described operations may serve to shift the prosthetic socket design performance testing from the first patient to the machine learning module 238 located on the computing 206 system so as to ease the physical burden on the first patient in finding an optimal prosthetic design fitment. To determine the prosthetic socket performance model for each iteration of prosthetic socket design created by the machine learning module 138, the machine learning module may leverage the gait data, optimal prosthetic socket data, and/or any other relevant data for one or more other patients. This data may be stored on the computing system. For example, the machine learning module may employ artificial intelligence (e.g., transfer learning) to determine how prosthetic socket designs created by the machine learning module 138 would perform on the first patient based on knowledge transferred from the data of the one or more other patients or the testing performed on the dummy residual limb and socket. The data from the one or more other patients and/or the data from testing performed on a dummy residual limb and socket may feed the machine learning module 138 and assist the machine learning module 138 in learning how prosthetic socket designs would perform based on different patient's residual limbs, and/or gait characteristics, and or any other relevant factors.

In various embodiments, at block 308, the method may involve providing, based on the third prosthetic socket design for the first patient, an output indicating an area of the first prosthetic socket design to add or remove material. In some embodiments, the filler material may be a foam-type material. The filler material may be inserted into the initial prosthetic socket to effectively convert the initial prosthetic socket to the optimized prosthetic socket for the first patient. In some embodiments, the output may indicate more than one area for inserting the filler material (and/or removing material).

### Initial Experimental Data Collection

FIG. 4 depicts a block diagram of an example testbed for testing a dummy residual limb and socket. In some embodiments, the testbed may include a frame 402 for holding the components of the testbed. Also included may be a mounting and swing mechanism 404 for mounting a dummy residual limb and socket 406. A load cell may 408 may be provided underneath the dummy residual limb and socket 406. Finally, a hydraulic loading mechanism 410 may be provided and configured to interact with the load cell 408. The hydraulic loading mechanism can also be replaced by adding weights along different directions to the dummy limb that is fixed in angled casted sockets with pressure sensors, and thereby the pressure can be estimated under different directional loads cost-effectively

In some embodiments, the testbed may be used to improve gait performance prediction and/or learning for a patient through testing of the dummy residual limb and socket. The usage of the testbed and dummy residual limb and socket may be necessary during initial implementation stages of the systems and methods described herein because there may be insufficient gait data, or other data, from other patients for use in the cloud computing system 106. However, testing using dummy residual limb and socket instead of a patient's actual limbs may be used even beyond the initial implementation stages. The use of the testbed may also eliminate the need to perform intensive testing on patients by performing the testing to the dummy residual limb and socket instead (providing loads onto the dummy prosthetic limb and obtaining resulting data). When more patients receive optimized sockets, we gradually build a database or library so that future patients with similar gait problems can be beneficial by leveraging the transfer learning

Gait experiments will first be developed and refined on dummy residual limb and socket to estimate the impact of prosthetic socket designs and interior stiffness on pressure distribution inside the socket. The dummy residual limb and socket may be created for individual patients seeking prosthetic limb prescriptions. Experiments and analyses will then be performed on a small population of amputee patients. Patients' discomfort will also be surveyed during testing. Once more patients receive optimized sockets, a database or library may be gradually developed and utilized by the transfer learning so that future patients with similar gait problems can be beneficial by leveraging the transfer learning.

The acquired gait and comfort data along with gait analyses will be integrated to establish a socket-gait progression model that predicts the gait given prosthetic socket designs/stiffness. This task develops a novel transfer learning algorithm that transfers the knowledge learned from dummy residual limb and socket made close to the patient or prior experiences to improve gait predictions given socket designs. A model will be used to optimize the prosthetic socket designs. Additive manufacturing will be used to produce different socket designs to validate their performance.

Although references are made to “prosthetic socket design,” or “prosthetic limb,” or the like, all of these and any similar terms may be used interchangeably. For example, a prosthetic socket design may also indicate the full prosthetic limb design.

Although specific embodiments of the disclosure have been described, numerous other modifications and alternative embodiments are within the scope of the disclosure. For example, any of the functionality described with respect to a particular device or component may be performed by another device or component. Further, while specific device characteristics have been described, embodiments of the disclosure may relate to numerous other device characteristics. Further, although embodiments have been described in language specific to structural features and/or methodological acts, it is to be understood that the disclosure is not necessarily limited to the specific features or acts described. Rather, the specific features and acts are disclosed as illustrative forms of implementing the embodiments. Conditional language, such as, among others, “can,” “could,” “might,” or “may,” unless specifically stated otherwise, or otherwise understood within the context as used, is generally intended to convey that certain embodiments could include, while other embodiments may not include, certain features, elements, and/or steps. Thus, such conditional language is not generally intended to imply that features, elements, and/or steps are in any way required for one or more embodiments

We claim:

1. A system comprising:
a processor; and
a memory storing computer-executable instructions, that when executed by the processor, cause the processor to:
receive data indicating a first prosthetic socket design for a first patient and data associated with the first patient;
receive data indicating a structure of an second prosthetic socket design for a second patient and data associated with the second patient;
determine, based on the data associated with the first patient, the data indicating the structure of an optimized prosthetic socket for the second patient, and the data associated with the second patient, an third prosthetic socket design for the first patient; and
provide, based on the third prosthetic socket design for the first patient, an output indicating an area of the first prosthetic socket design to add or remove material.

2. The system of claim 1, wherein the computer-executable instructions further cause the processor to:
receive data indicating a structure of a fourth prosthetic socket design for a third patient and data associated with the third patient;
determine, based on the data indicating a structure of the fourth prosthetic socket design for the third patient, and the data associated with the third patient, a fifth prosthetic socket design for the first patient; and
provide, based on the fifth prosthetic socket design for the first patient, an output indicating a change to the third prosthetic socket design to result in the fifth prosthetic socket design, the change including at least one of: adding or removing material from the third prosthetic socket design.

3. The system of claim 1, wherein the computer-executable instructions further cause the processor to:
provide, based on the optimized prosthetic socket for the first patient, a data model of the third prosthetic socket design to a 3D printing device for printing of the optimized prosthetic socket.

4. The system of claim 1, wherein the computer-executable instructions further cause the processor to receive data regarding an interaction between the first prosthetic socket based on the first prosthetic socket design and the first patient, the interaction including at least one of: a fitment of a residual limb of the first patient relative to the first prosthetic socket or pressure between the first prosthetic socket and the residual limb at one or more points.

5. The system of claim 1, wherein the computer-executable instructions further cause the processor to:
receive data regarding an interaction between a second prosthetic socket based on the third prosthetic socket design and the first patient; and
provide, based on the interaction, an indication of a fifth prosthetic socket design for the first patient.

6. The system of claim 1, wherein the material is a foam material.

7. The system of claim 1, wherein the data associated with the first patient and the data associated with the second patient include a gait of the first patient and a gait of the second patient respectively.

8. A method comprising:
receiving data indicating a first prosthetic socket design for a first patient and data associated with the first patient;
receiving data indicating a structure of an second prosthetic socket design for a second patient and data associated with the second patient;
determining, based on the data associated with the first patient, the data indicating the structure of the optimized prosthetic socket for the second patient, and the data associated with the second patient, an third prosthetic socket design for the first patient; and
providing, based on the third prosthetic socket design for the first patient, an output indicating an area of the first prosthetic socket design to add or remove material.

9. The method of claim 8, further comprising:
receiving data indicating a structure of a fourth prosthetic socket design for a third patient and data associated with the third patient;
determining, based on the data indicating a structure of the fourth prosthetic socket design for the third patient, and the data associated with the third patient, a fifth prosthetic socket design for the first patient; and
providing, based on the fifth prosthetic socket design for the first patient, an output indicating a change to the third prosthetic socket design to result in the fifth prosthetic socket design, the change including at least one of: adding or removing material from the third prosthetic socket design.

10. The method of claim 8, further comprising:
providing, based on the optimized prosthetic socket for the first patient, a data model of the third prosthetic socket design to a 3D printing device for printing of the optimized prosthetic socket.

11. The method of claim 8, further comprising:
receiving data regarding an interaction between the first prosthetic socket and the first patient, the interaction including at least one of: a fitment of a residual limb of the first patient relative to the first prosthetic socket or pressure between the first prosthetic socket and the residual limb at one or more points.

12. The method of claim 8, further comprising:
receiving data regarding an interaction between a second prosthetic socket based on the third prosthetic socket design and the first patient; and
providing, based on the interaction, an indication of a fifth prosthetic socket design for the first patient.

13. The method of claim 8, wherein the material is a foam material.

14. The method of claim 8, wherein the data associated with the first patient and the data associated with the second patient include a gait of the first patient and a gait of the second patient respectively.

15. A non-transitory computer readable medium including computer-executable instructions stored thereon, which when executed by one or more processors of a wireless access point, cause the one or more processors to perform operations of:
receiving data indicating a first prosthetic socket design for a first patient and data associated with the first patient;
receiving data indicating a structure of an second prosthetic socket design for a second patient and data associated with the second patient;
determining, based on the data associated with the first patient, the data indicating the structure of the optimized prosthetic socket for the second patient, and the data associated with the second patient, an third prosthetic socket design for the first patient; and
providing, based on the third prosthetic socket design for the first patient, an output indicating an area of the first prosthetic socket design to add or remove material.

16. The non-transitory computer readable medium of claim 15, wherein the computer-executable instructions further cause the processor to:
receiving data indicating a structure of a fourth prosthetic socket design for a third patient and data associated with the third patient;
determining, based on the data indicating a structure of the fourth prosthetic socket design for the third patient, and the data associated with the third patient, a fifth prosthetic socket design for the first patient; and
providing, based on the fifth prosthetic socket design for the first patient, an output indicating a change to the third prosthetic socket design to result in the fifth prosthetic socket design, the change including at least one of: adding or removing material from the third prosthetic socket design.

17. The non-transitory computer readable medium of claim 15, wherein the computer-executable instructions further cause the processor to:
providing, based on the optimized prosthetic socket for the first patient, a data model of the third prosthetic socket design to a 3D printing device for printing of the optimized prosthetic socket.

18. The non-transitory computer readable medium of claim 15, wherein the computer-executable instructions further cause the processor to:
receiving data regarding an interaction between the first prosthetic socket and the first patient, the interaction including at least one of: a fitment of a residual limb of the first patient relative to the first prosthetic socket or pressure between the first prosthetic socket and the residual limb at one or more points.

19. The non-transitory computer readable medium of claim 15, wherein the computer-executable instructions further cause the processor to:
receiving data regarding an interaction between a second prosthetic socket based on the third prosthetic socket design and the first patient; and
providing, based on the interaction, an indication of a fifth prosthetic socket design for the first patient.

20. The non-transitory computer readable medium of claim 15, wherein the data associated with the first patient and the data associated with the second patient include a gait of the first patient and a gait of the second patient respectively.
